(19) United States
(12) Patent Application Publication
(10) Pub. No.: US 2013/0149362 A1
(43) Pub. Date: Jun. 13, 2013

(54) TREATMENT PRODUCT AND METHOD

(71) Applicant: Benjamin Johnson, Evergreen, CO (US)

(72) Inventor: Benjamin Johnson, Evergreen, CO (US)

(21) Appl. No.: 13/758,954

(22) Filed: Feb. 4, 2013

Related U.S. Application Data
(63) Continuation-in-part of application No. 11/686,883, filed on Mar. 15, 2007.

(51) Int. Cl. A61K 8/14; A61Q 19/00; A61K 8/67; A61K 9/127; A61K 31/11

(52) U.S. Cl. CPC A61K 8/14; A61K 9/127; A61K 31/11; A61K 8/671; A61Q 19/00
USPC 424/401; 424/450; 514/703

(57) ABSTRACT

Treatment products and methods are disclosed. An example treatment product may be used for treating skin disorders by stimulating the dermis and epidermis skin layers. The example treatment product may have a composition of retinal in the amount of at least 0.15% by weight, and a liposome surrounding the retinal.

This application is a continuation-in-part of U.S. patent application Ser. No. 11/686,883 filed Mar. 15, 2007 entitled “Treatment of Dermatologic Skin Disorders” of Benjamin Johnson, which is hereby incorporated by reference in its entirety.

### BACKGROUND

Facial skin as well as other skin areas often suffers damage from exposure to sunlight, heat, cold or wind, or from hyperpigmentation, or from skin disorders such as precancerous neoplasm, or from other sources. Skin also undergoes wrinkling or other aging processes. Acne and other skin disorders may also scar or disfigure skin as well. Further damage may result from injuries that may cause scarring or other disfigurement. These skin disorders are often a source of concern for many individuals.

Many skin disorders, such as facial blemishes, wrinkles, uneven skin pigmentation, acne facials scars, precancerous skin growths and others, are often treated by skin peels. While skin peels may include mechanical removal, dermabrasion, carbon and laser treatments, the most popular are chemical peels. Chemical peels use a chemical solution to remove damaged outer layers of skin to improve and smooth the texture of the facial skin. The chemical solution causes the damaged skin to blister and eventually peel off.

There are several well-known chemical peel treatments for skin disorders. These are typically categorized as superficial, medium and deep depending on the depth of the skin wounding that occurs. Superficial peels are used to treat fine wrinkles, areas of dryness, areas of skin irritation, uneven pigmentation and acne. These peels remove or effect replacement or replenishment of the epidermis skin layer. Medium peels penetrate to the papillary dermis layer. These peels are often used to treat fine surface wrinkles superficial blemishes, pigment problems and other deeper skin disorders. Deep peels penetrate to the reticular dermis. These are often used to treat coarse facial wrinkles, areas of blotch or damaged skin caused by sun exposure or pre-cancerous growths and for lightening purposes.

Superficial peels typically use alphahydroxy acids (AHAs) such as glycolic, lactic or fruit acids, trichloroacetic acid (TCA) in smaller concentrations, and Jessner's solution. Superficial peels are often used for individuals who do not want to have to recover from a medium or deep peel, and for maintenance purposes. These peels do have issues relating to their lack of effectiveness, stinging, skin irritation, dryness and the need for multiple treatments.

Medium peels typically use 40-50% trichloroacetic acid (TCA). These peels are able to correct disorders that are beyond the skin surface. Problems with these peels include the need for pretreatment with AHA creams, repeat treatments, the need for sun protection for several months, a greater degree of pain and discomfort and the need for healing periods of several days.

Deep peels typically use phenol as a chemical solution. These are the strongest of the chemical solutions and are the longest lasting. Typically, a deep phenol peel requires only a single treatment to achieve the desired result. Deep peels do have issues including posing a risk for patients with heart problems, removing facial freckles, reducing the skin's ability to produce pigment, thus preventing the skin from tanning, requiring increased protection from the sun for life, causing permanent skin lightening, and taking several months to heal. Deep peels are very painful and usually require general anesthesia or very heavy sedation.

All of these chemical peels use some form of acid to bum the skin to achieve the desired results. This causes trauma to the skin, at the very least, stinging and irritation, and in medium and deep peels, pain and pigmentation loss. These chemical peels may even cause damage to the health of the skin to achieve the desired results. It is often necessary to use additional compositions along with these acids to treat the post-traumatic side effects from the use of these acid chemical peels.

### DETAILED DESCRIPTION

The treatment product and method disclosed may be provided as a composition that penetrates the layers of skin to stimulate the epidermis and dermis. This results in exfoliation of the skin to remove damages skin as well as improving the health of the skin. The treatment product and method may use retinal, retinal derivatives as well as any other retinoids, and may further include liposomes. The example treatment product and method may be used to increase the efficacy of the use of retinal for therapeutically and cosmetically treating many skin disorders.

In an example, the treatment product and method increase the efficacy of a topical skin care product by increasing the penetration of retinal or its derivatives into the skin. Compositions containing retinal or its derivatives are coated or mixed with liposomal materials. The liposomal retinal compound has been shown to increase the penetration of the retinal or retinal derivatives by ten fold which not only increases the efficacy of the retinal product but also reduces the amount of retinal in the superficial skin layers. This greatly reduces the irritation in these layers as well as increasing the moisture in the skin.

In another example, the treatment product and method reduce the dehydration of the skin from using retinal or its derivatives. Compositions containing retinal or its derivatives are coated or mixed with liposomal materials. Liposomes have a natural affinity for water which assists in increasing the moisture in the skin during topical application of the liposomal retinal composition. Also, as discussed above, the liposomal retinal composition has increased penetration which reduces the retinal in the superficial skin layer which in tum decreases the dehydration of the skin.

In an example, the treatment product and method reduce the irritation to the skin in using retinal or retinal derivatives. Compositions containing retinal or its derivatives are coated or mixed with liposomal materials. The reduction of retinal in the superficial skin layers by the deeper penetration of retinal using the liposomal formulation also decreases the irritation of the skin. Also the additional hydration of the skin using the liposomal formulation reduces the irritation as well.

In an example, the treatment product and method utilize liposomal retinal compositions as a chemical peel. Compositions containing retinal or its derivatives are coated or mixed with liposomal materials. The composition is used as strong stimulant of the dermis as well as promoting temporary exfoliation to remove areas of skin layers that may be damaged. The increased penetration of the liposomal retinal composition increases the efficacy of the chemical peel while also reducing the dehydration and irritation of the process. This also allows this chemical peel to affect the skin without using harsh acids which is the primary cause of unwanted side effects like post-inflammatory hyperpigmentation and scarring.

In another example, the treatment product and method provide retinal in a chemical peel treatment. The retinal is provided in a strength of at least 0.15% by weight. In an example, the retinal is provided in a liposomal material to increase the penetration rate of the retinal. The liposomal retinal is able to penetrate sufficiently to stimulate the dermis and epidermis skin layers. The damaged skin is exfoliated and the health of the skin is improved.

The chemical peel treatment is mild, yet effective. In an example, the pH level of this example is about 2 or greater, for example in the range of 2 to 7. In another example, the pH level is about 3.0 to 3.5. This results in the skin not being burned and little or no discomfort is incurred by the patient.

Before continuing, it is noted that as used herein, the terms “includes” and “including” mean, but is not limited to, “includes” or “including” and “includes at least” or “including at least.” The term “based on” means “based on” and “based at least in part on.”

The example treatment product and method provide products and method for treating skin disorders. It is to be expressly understood that this example is provided for descriptive purposes only and is not meant to unduly limit the scope of the present inventive concept. Other examples of the skin care products and method of use of the example treatment product and method are considered within the present inventive concept as set forth in the claims herein. For explanatory purposes only, the skin care products and method of use of the example are discussed primarily with the use of substantially acid-free chemical compositions. It is to be expressly understood that other products and method are contemplated for use with the example treatment product and method as well with combinations of acids as well.

Retinal and its derivatives may be incorporated with liposomes in therapeutic compositions for treating skin disorders. In particular, retinal and its derivatives are combined with liposomes for use as a chemical skin peel. This substantially acid-free skin peel stimulates the epidermis and dermis which results in exfoliation and improvement to the health of the skin. The use of the liposomal retinal also increases the penetration of the retinal into the skin, provides hydration of the skin during treatment, and other benefits. The example treatment product and method include utilizing retinol products, and particularly retinal in combination with liposomes each of these examples and uses separately as well as in combinations with one another. While the example discusses an substantially acid-free chemical peel, it is to be expressly understood that the claims also encompasses examples using acids as well.

The example treatment product and method utilize retinol products and in particular retinal and retinal derivatives such as those described in U.S. Pat. No. 5,492,935 and 5,093,360 which are incorporated herein by reference and further described below. The treatment product and method is described below as examples utilizing retinal and retinal derivatives. However, it is to be expressly understood that other forms of retinal and other retinol products are considered to be within the scope of the claims.

Retinal which is also often called vitamin A aldehyde differs considerably from retinol and Retinoic acid. Retinol which is often called vitamin A alcohol and vitamin A is present in fish liver oils as an ester compound, retinyl palmitate which is used in many skin care products. Retinoic acid, also called vitamin A acid, is an oxidation product from retinol or retinal. Retinol and retinal are critical in the physiologic functions of vision, growth, reproduction and differentiation. Retinoic acid on the other hand does not contribute to vision and reproduction in humans and animals.

Retinal can exist in stereoisomeric forms, namely all-trans, 13-cis, 11-cis, 9-cis, 7-cis, 11,13-cis, 9,13-cis. However, the common form is all-trans retinal. Since retinal is chemically an aldehyde it can exist as hemiacetal and acetal forms by reacting with one or two molecules of an alcohol, such as methanol, ethanol or propanol. Such hemiacetal and acetal forms are usually more stable against alkali, and are more resistant to oxidation of the aldehyde group. Retinal may be shown by the following chemical structure: R₁, R₂ .dbd.alkyl, aralkyl, aryl or alkoxy group of saturated or unsaturated, straight or branched chain or cyclic form, having 1 to 25 carbon atoms, R₃ .dbd.O or (OR₄)(OR₅), wherein R₄, R₅ .dbd.H, alkyl, aralkyl, aryl, diol or polyol group of saturated or unsaturated, straight or branched chain or cyclic form, having 1 to 25 carbon atoms; and the hydrogen atom attached to the carbon atom in the main chain as well as in R₁, R₂, R₄ and R₅ may be substituted by a halogen atom or a radical such as a lower alkyl, aralkyl, aryl or alkoxy having 1 to 9 carbon atoms.

The hemiacetal and acetal forms are represented by CH(OR₄)(OR₅) instead of —CHO. The compound may be called retinal hemiacetal when either R₄ or R₅ is H, and the compound is called retinal acetal when both R₄ and R₅ are for example alkyls or aralkyls. The compound is called retinal hydrate when both R₄ and R₅ are H.

Retinal and its derivatives may exist as stereoisomers such as all-trans, 13-cis, 11-cis, 9-cis, 7-cis, 11,13-cis and 9,13-cis forms.

The typical alkyl, aralkyl and aryl groups for R₁ and R₂ are for example, methyl, ethyl, propyl, isopropyl, butyl, pentyl, octyl, lauryl, stearyl, benzyl and phenyl etc. The halogen atoms are F, Cl, Brand I. The typical alkoxy groups are methoxy and ethoxy. The typical diol groups are glycol, propylene glycol and 1,3-butanediol. The typical polyol groups include glycerol, butanetriol, inositol and alditols; such as erythritol of tetraol, ribitol of pentaol, mannitol and sorbitol of hexaols.

Retinal or its derivative may react with an unsaturated chemical agent such as maleic anhydride, acetylene dicarboxylic acid or its ester, or hydroquinone to form a crystalline molecular complex called an adduct compound.

The representative retinal and its derivatives which may be useful for topical or systemic administration to improve cosmetic conditions or to alleviate dermatologic disorders are listed below:

All Trans Retinal; Retinal hydrate; Retinal methyl hemiacetal; Retinal ethyl hemiacetal; Retinal propyl hemiacetal; Retinal isopropyl hemiacetal; Retinal butyl hemiacetal; Retinal pentyl hemiacetal; Retinal octyl hemiacetal; Retinal benzyl hemiacetal; Retinal dimethyl acetal; Retinal diethyl acetal; Retinal dipropyl acetal; Retinal diisopropyl acetal; Retinal dibutyl acetal; Retinal dipentyl acetal; Retinal dioctyl acetal; Retinal dibenzyl acetal; Retinal hydroquinone adduct; Retinal maleic anhydride adduct; Retinal acetylene dicarboxylic acid ester adduct; Retinal propylene glycol hemiacetal and acetal; Retinal 1,2-o-isopropylidene glyceryl hemiacetal and acetal; Retinal 3-allyloxy-1,2-propanediol hemiacetal and acetal; Retinal phytyl hemiacetal; Retinal diphytyl acetal; Retinal dodecyl hemiacetal; and Retinal didodecyl acetal.

Retinal and its derivatives may also be utilized in combination with or as additives to enhance therapeutic effects of other cosmetic or pharmaceutical agents to improve cosmetic conditions or alleviate the symptoms of dermatologic disorder. Cosmetic and pharmaceutical agents include those that improve or eradicate age spots, keratoses and wrinkles eradicating agents; antiacne agents; antibacterials; antiyeast agents; antifungal agents; antiviral agents; antidandruff agents; antidermatitis agents; antipruritic agents; antiinflammatory agents; antihyperkeratolytic agents; antidryskin agents; antipsoriatic agents; antiseborrheic agents; hair conditioners and hair treatment agents; antiaging and antiwrinkle agents; sunscreen agents; antihistamine agents; vitamins; corticosteroids, tanning agents; local anesthetics; hormones; retinoids and other dermatologicals.

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

Accordingly, an example treatment product and method provides cosmetic as well as medicinal compositions containing retinal or its derivatives coated in liposomal material which when topically or systemically administered will substantially improve and alleviate the symptoms of various cosmetic conditions or dermatologic disorders.

Another example treatment product and method provides method for treating various cosmetic conditions or dermatologic disorders with topical preparations containing retinal or its derivatives in conjunction with liposome material to improve penetration of the retinal into the skin, provide hydration during the application of the retinal or its derivatives, increase the efficacy of the retinal or its derivatives and/or to reduce oxidation and irritation normally seen with retinal in topical formulations.

Retinal and its derivatives may be formulated for topical application. In example topical preparations, retinal and its derivatives are formulated so the composition contains 0.0001 to 5%, and in an example from about 0.2% to about 5%, and in another example about 2% by weight of the total composition.

If retinal and its derivatives are formulated in aqueous form, sodium sulfite, sodium bisulfite, sodium metabisulfite or other antioxidants may be added to stabilize retinal and its derivatives in aqueous compositions. Liposomal lecithin or a liposome substitute or other lipid preparations are added to the above solution with mixing until a uniform consistency is obtained. Butylated hydroxytoluene (BHT) or butylated hydroxyanisole (BHA) may be added to stabilize retinal and its derivatives in non-aqueous composition. To provide maximal stability of the therapeutic compositions antioxidants of both aqueous and nonaqueous types may also be incorporated into the compositions at the same time. For example, both sodium metabisulfite and BHT may be added to an aqueous acoholic solution containing retinal. The concentration of antioxidant may range from 0.01 to 5%. The most efficacious stabilizer is a liposomal coating which provides a lipid layer of protection and the concentration may range from 0.001 to 10%.

To prepare a typical aqueous solution, retinal or its derivative is dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, retinal or its derivative is dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved. When a combination composition is desired retinyl palmitate and/or hydroquinone, for example is added to the above non-aqueous solution. The concentration of retinyl palmitate ranges from about 1 to 5%. The concentration of hydroquinone may range from about 1 to 5%, in an example the concentration is 2% by weight of the total composition.

A typical cream or lotion containing retinal or its derivative is prepared by first dissolving retinal or its derivative in ethanol, acetone, propylene glycol or other solvent. The solution thus prepared is then admixed with commonly available oil-in-water emulsions. BHT or sodium metabisulfite may be added to such emulsions to stabilize retinal or its derivative. Liposomes or liposome substitutes are added to this solution with mixing until a uniform consistency is achieved.

A typical gel composition is formulated by first dissolving retinal or its derivative in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The concentration of the gelling agent may range from about 0.2 to 2% by weight of the total composition. Liposomes or liposome substitutes are added to this solution with mixing until a uniform consistency is achieved.

The above examples of formulations and compositions of descriptive examples are provided as a general explanation. It is expressly noted that these examples are intended to be illustrative and not limiting.

The example treatment product and method provides liposomal retinal at strengths greater than 0.15% by weight. This provides a mild but highly effective peel that improves the health of the skin. The resulting product of this example has a pH above about 2 with little to no trauma to the skin with the application of this product.

An example treatment product and method increases the efficacy of a topical skin care product by increasing the penetration of retinal or its derivatives into the skin. Retinal by itself has a penetration rate that is as low as about 2%. Compositions containing retinal or its derivatives are coated or mixed with liposomal materials as described above. The liposomal retinal compound has been shown to increase the penetration of the retinal or retinal derivatives by ten fold which not only increases the efficacy of the retinal product but also reduces the amount of retinal in the superficial skin layers.

This example reduces the dehydration of the skin from using retinal or its derivatives. Compositions containing retinal or its derivatives are coated or mixed with liposomal materials as described above. Liposomes have a natural affinity for water which assists in increasing the moisture in the skin during topical application of the liposomal retinal composition. Also, as discussed above, the liposomal retinal composition has increased penetration which reduces the retinal in the superficial skin layer which in turn decreases the dehydration of the skin.

Another example utilizes liposomal retinal compositions as a chemical peel. Compositions containing retinal or its derivatives are coated or mixed with liposomal materials as described above with retinal provided in the strengths of greater than about 0.15% by weight. The composition is used as chemical peel to remove areas of skin layers that may be damaged. The increased penetration of the liposomal retinal composition increases the efficacy of the chemical peel while also reducing the dehydration and irritation of the process.

### Examples

The following examples are illustrative of specific treatment products for treating skin disorders. These examples are not intended to be limiting.

An example treatment product may be used for treating skin disorders by stimulating the dermis and epidermis skin layers.

An example treatment product having a pH 3.4 includes Aqua (Water), Shebu, Phosphatidylcholine, Niacinamide, Mandelic Acid (L), Alcohol Denat., Retinal (Retinaldehyde), Sodium Hyaluronate (L), Lactic Acid (L), Glycerin, Chlorella Vulgaris Extract, Thioctic (R-lipoic) Acid, Panthenol (D), Pyridoxine HCl, Dimethyl Sulfone, Beta-Glucan (D), Fulvic Acid, Hippophae Rhamnoides (Sea Buckthorn) Extract, Chrysanthemum Parthenium (Feverfew) Extract, Epilobium Angustifolium Flower/Leaf/Stem Extract, Cymbopogon Martini (Palmarosa) Oil, Santalum Austrocaledonicum (Sandalwood) Wood Oil, Tocopherol (D-alpha), Glycine Soja (Soybean) Oil, Lonicera Caprifolium (Honeysuckle) Flower Extract, Lonicera Japonica (Honeysuckle) Flower Extract, Magnesium Chloride, Alcohol, Xantham Gum, Benzyl Alcohol, Potassium Sorbate.

The treatment product may have a composition including: a retinal in the amount of at least 0.15% by weight; and a liposome surrounding the retinal.

An effective peel may need delivery of a sufficient amount of retinal beneath the epidermis. It has been found that the liposome may be provided in an amount of 2-10% by weight. Liposomal delivery, e.g., using phosphatilycholine increases delivery of the retinal.

The retinal may be in the amount of at least 0.15% by weight. In some applications, the retinal may not be an effective peel. For example, if the retinal is unstable, very little active ingredient will be left to accomplish the skin peel. It has been found that the retinal may be provided as retinaldehyde. Retinaldehyde has been found to be effective in this regard when provided in an amount of about 2% by weight.

The treatment product may further include a polyethylene glycol (PEG). The PEG may be provided in an amount of about 0.1-20% by weight. In an example, the PEG is PEG-50 Shea Butter commercially available under the trade name Shebu in an amount of 5-20% by weight. The PEG may be used to help stabilize the composition.

The composition may have a pH of about 2-7. In an example, the treatment product may not have any acids. In another example, the composition includes an acid.

The example treatment product and method may be embodied in other specific forms without departing from the spirit or essential characteristics thereof. The examples are to be considered in all respects as illustrative and not restrictive, the scope of the claims including all changes which come within the meaning and equivalency of the recitations therein are therefore intended to be embraced therein.

For example, the above described example eliminates acids from use as a skin peel. In other examples within the scope of the claims, the peel treatment may also use acids in combination with liposomes and/or retinal. Other combinations may be used as well to promote other properties within the scope of the claims.

It is noted that the examples shown and described are provided for purposes of illustration and are not intended to be limiting. Still other examples are also contemplated.

1. A chemical peel method for treating skin disorders, the method comprising:
providing a retinal within a liposome in a composition; and
exfoliating the skin by applying the composition to the skin disorder to stimulate the dermis and epidermis layers.

2. The method of claim 1, further comprising providing retinal in the amount of at least 0.15% by weight.

3. The method of claim 1, further comprising providing the retinal as retinaldehyde in an amount of about 2% by weight.

4. The method of claim 1, wherein providing the liposome is in an amount of 2-10% by weight.

5. The method of claim 1, further comprising providing a polyethylene glycol (PEG).

6. The method of claim 5, further comprising providing the PEG in an amount of about 0.1-20% by weight.

7. The method of claim 6, further comprising providing the PEG as PEG-50 Shea Butter.

8. The method of claim 7, further comprising providing the PEG-50 Shea Butter in an amount of 5-20% by weight.

9. The method of claim 1, further comprising providing the composition with a pH in the range of about 2-7.

10. The method of claim 1, further comprising providing the composition with substantially no acids.

11. The method of claim 1, further comprising providing the composition with an acid.

12. A treatment product for treating skin disorders by stimulating the dermis and epidermis skin layers, the chemical peel treatment product having a composition comprising:
a retinal in the amount of at least 0.15% by weight; and
a liposome surrounding the retinal.

13. The treatment product of claim 12, further comprising the retinal in the amount of at least 0.15% by weight.

14. The treatment product of claim 12, wherein the retinal is retinaldehyde in an amount of about 2% by weight.

15. The treatment product of claim 12, wherein the liposome is in an amount of 2-10% by weight.

16. The treatment product of claim 12, further comprising a polyethylene glycol (PEG) in an amount of about 0.1-20% by weight.

17. The treatment product of claim 12, further comprising PEG-50 Shea Butter in an amount of 5-20% by weight.

18. The treatment product of claim 12, wherein the composition has a pH of about 3.4.

19. The treatment product of claim 12, wherein the composition has substantially no acids.

20. The treatment product of claim 12, wherein the composition includes an acid.
